(19) United States
(12) Patent Application Publication
(10) Pub. No.: US 2016/0367314 A1
(43) Pub. Date: Dec. 22, 2016

(54) BENDABLE AND REBENDABLE ENDOSCOPIC ELECTROSURGICAL DEVICE

(71) Applicant: Tenjin LLC, Brazoria, TX (US)

(72) Inventors: Christopher P. DOUGHERTY, Rogers, AR (US); Robert A. VAN WYK, St. Pete Beach, FL (US); Gary R. HEISLER, Brazoria, TX (US)

(73) Assignee: TENJIN LLC, Brazoria, TX (US)

(21) Appl. No.: 15/252,873

(22) Filed: Aug. 31, 2016

Related U.S. Application Data
(63) Continuation-in-part of application No. 14/635,266, filed on Mar. 2, 2015.
(60) Provisional application No. 61/966,739, filed on Feb. 28, 2014.

(51) Int. Cl. A61B 18/14; A61B 18/12; A61B 17/00

(52) U.S. Cl. CPC A61B 18/1492; A61B 17/00234; A61B 18/1233; A61B 2018/00577

(57) ABSTRACT

In the context of endoscopic electrosurgery, there is a need for a sufficiently robust, rigid, and preferably aspirating electrosurgical device that may be repeatedly flexed in the field to allow access to a wide array of remote tissues using a single device and, more preferably, during a single minimally invasive procedure. The present invention addresses this significant need by providing the distal portion of the elongate tubular member of an electrosurgical device with a bend region having a non-uniform flexural strength along its length, such that one may bend the tubular member at said bend region not only to an initial small radius bend, but subsequently rebend the distal portion to other angles as needed, with all bends occurring in the same distal region. A number of different mechanisms for reducing the flexural strength in the bend region are disclosed herein, including, for example, notching the tube in the bend region, annealing the tube in the bend region, reducing the wall thickness in the bend region, utilizing a material having lower flexular and/or yield strength to form the bend region, utilizing a small diameter elongate conductive element having negligible rigidity, such as a wire, and combinations thereof.

### PRIORITY

This application is a continuation-in-part of U.S. application Ser. No. 14/635,266 filed Mar. 2, 2015, which, in turn, claims the benefit of U.S. Provisional Application No. 61/966,739 filed Feb. 28, 2014, the contents of each of which are hereby incorporated by reference in their entirety.

### TECHNICAL FIELD OF THE INVENTION

The present invention relates generally to the field of endoscopic surgery and electrosurgical instruments, particularly radio frequency (RF) surgical instruments, for use therein. More particularly, the invention relates to a minimally invasive endoscopic electrosurgical device having a distal portion that may be readily flexed or bent by the surgeon to a first angle to access a first target tissue and then rebent to a second angle to access a second target tissue, with both bends occurring at a predetermined location on the device.

### BACKGROUND OF THE INVENTION

Accessing certain locations for treatment during certain minimally invasive procedures, such as endoscopy, arthroscopy and laparoscopy, can be difficult for surgeons. Radio frequency devices for the bulk vaporization of tissue, commonly referred to in the art as “ablators”, having an angularly offset distal portion that allows a surgeon to access portions of the anatomy not readily reached with standard unbent ablators, are well known in the art. Examples of these include the CoVac 70, CoVac 50, TriStar 50, Titan 80 and others by Arthrocare, Inc., (Austin, Tex.). The distal portions of these devices are formed to the desired angular offset during manufacture, wherein bending fixtures and dies are used to produce repeatable bends with small radii. Other ablator devices have an articulating distal portion that allows the surgeon to change the angle of the distal portion inside the joint space or other body cavity. Examples of these include the SideWinder Blade ICW device, also by Arthrocare, the Eflex Electrothermal Probes by Smith and Nephew (Andover, Mass.), and the NavX device by Arthrex (Naples, Fla.). These devices, however, lack rigidity and moreover, due to their complex construction, may break during use. In addition, they are not designed to aspirate ablation byproducts from the site.

Aspirating arthroscopic RF ablators (for bulk tissue vaporization) tend to be supplied either as straight, pre-bent or articulating, but not bendable in the field. The elongate tubular section that makes up the distal portion of a conventional arthroscopic ablator has generally uniform structural properties throughout its length. In the case of pre-bent ablators, bending of the distal portion of the tubular section during manufacture allows the use of dies and other tooling that are able to repeatably produce bends having a small radius. Attempting to modify the angular offset of such a pre-bent blade would result not in modification of the original bend, but in bending at locations on the tubular member proximally adjacent to the bend produced during manufacture. Furthermore, bending of the tube during manufacture work-hardens the material in the bent region so that an attempt to modify the bend after manufacture will cause adjacent regions that have not been work-hardened to deform. While producing a bend in a straight ablator in the field may be possible in some instances (though generally against the manufacturer's recommendations), the bend will have a large radius and will moreover be well removed from the distal portion of the ablator. Attempting to rebend such a blade, particularly to a shallower angle, will result in further distortion of the tubular portion since the original bend will have work hardened the material in the bend region.

Arthrocare Corporation (Austin, Texas) manufactures bendable ablation devices for removal of the tonsils and adenoids. The Arthrocare EVac Plasma Wands are furnished to the surgeon unbent and used in that form to remove tonsils. When the tonsillectomy is complete, the distal portion of the device is bent to approximately 60 degrees using the EVac Bending Tool so that the surgeon can access the adenoids while indirectly visualizing the site with a mirror positioned in the patient's oral cavity. However, the device can only be bent once and is discarded when the adenoidectomy is complete.

Arthrocare Corporation also produces bendable endoscopic devices for the thermal treatment of tissue. The Arthrocare CAPsure and MicroCAPs devices may be bent using the Arthrocare Bending Tool so as to angularly offset their distal portions. However, these devices are for the thermal treatment of tissue only and are thus incapable of tissue vaporization or cutting. Critically, neither Arthrocare Corporation nor its competitors produce an endoscopic vaporization device that is bendable.

Accordingly, there is a clear need in the endoscopic arts for a sufficiently robust, rigid, and optionally aspirating electrosurgical device that may be repeatedly flexed in the field to allow access to a wide array of remote tissues using a single device. The present invention addresses this significant need by providing an improved bendable and rebendable endoscopic electrosurgical device that may be formed, flexed, or bent by the surgeon during use to a first desired configuration adapted to reach a first target tissue and then reformed or rebent to a second configuration when a different angle is required to access a second target tissue.

### SUMMARY OF THE INVENTION

Central to the present invention is the discovery that by providing the elongate distal tubular member of an electrosurgical device with a non-uniform flexural strength along its length, one may then bend the tubular member at distal locations not only to an initial small radius bend, but further to other angles as needed, with all bends occurring in the same distal region. Specifically, in a particularly preferred embodiment, the flexural strength of a least a portion of the tubular member near its distal end is reduced such that an initial small-radius bend and angular offset may be produced in the tubular member by the surgeon as required. Thereafter, the surgeon can subsequently, for example, through the use of a manual bending device, modify the angular offset of the tubular member to form a different bend adapted to access other remote target tissue sites. The angular offset of the tubular member's distal end may be modified by manipulating the degree of bend, with the deformation of the tubular member remaining localized in the bend region since adjacent portions of the tubular member's have a higher flexural strength. The flexural strength in the bend region may be reduced by any number of different mechanisms, for example, by notching or slicing the tube in the bend region, by annealing the tube in the bend region, by reducing the wall thickness in the bend region, by utilizing a material having lower flexular and/or yield strength to form the bend region or by any combination of these means.

Accordingly, it is an objective of the present invention to provide a bendable and rebendable electrosurgical device composed of a proximal handle portion assembled to a distal elongate tubular member that defines the longitudinal axis of the device, wherein the elongate tubular member includes:
a. a proximal end configured to the distal end of the handle portion;
b. a distal end including an active electrode; and
c. a distal portion including a bend region disposed in close proximity to the distal end that comprises a length of reduced flexural strength relative to that of the remainder of the elongate tubular member, wherein the bend region may be repeatedly flexed away from the longitudinal axis, in the longitudinal or lateral direction.
   In the context of the present invention, the repeated flexing optionally includes (a) bending the distal portion to a first angle, and (b) rebending the distal portion to a second angle, wherein the first angle enables contact of the active electrode to a first set of target tissues and the second angle enables contact of the active electrode to a second set of target tissues. In the context of the present invention, the first and second angles differ from each other, either in terms of size or direction or both.

In a preferred embodiment, the bendable and rebendable electrosurgical device operates in a bipolar fashion by further including an insulating polymeric sleeve or dielectric coating extending over the elongate tubular member, from its proximal end to just past the bend region, thereby defining an uninsulated distalmost region disposed between the distal end of the bend region and the active electrode that acts as a return electrode. In the context of the present invention, the elongate tubular member is preferably fabricated from a conductive material such as metal.

It is a further object of the present invention to provide a kit for bending and rebending an electrosurgical device that includes the bendable and rebendable electrosurgical device described above in combination with (b) an external bending tool.

It is yet another object of the present invention to provide a method of performing electrosurgery in a subject in need thereof, the method including the steps of:
(a) providing the bendable and rebendable electrosurgical device as described above;
(b) bending the distal portion in a first direction, to a first angle, whereby the active electrode contacts a first set of target tissue; and
(c) rebending the distal portion in a second direction, to a second angle, whereby the active electrode contacts a second set of target tissues.

These and other aspects are accomplished in the invention herein described, directed to a uniquely flexible, bendable and re-bendable endoscopic surgical device. Further objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention. In particular, while the invention is described herein with reference to a number of specific embodiments, it will be appreciated that the description is illustrative of the invention and is not constructed as limiting of the invention.

### BRIEF DESCRIPTION OF THE FIGURES

Various aspects and applications of the present invention will become apparent to the skilled artisan upon consideration of the brief description of figures and the detailed description of the present invention and its preferred embodiments that follows:

FIG. 1 is a plan view of the tubular member for an arthroscopy ablator formed in accordance with the principles of this invention.

FIG. 2 is a side elevational view of the objects of FIG. 1.

FIG. 3 is an expanded view of the distal portion of the objects of FIG. 1 at location A of FIG. 1.

FIG. 4 is an expanded view of the distal portion of the objects of FIG. 2 at location B of FIG. 2.

FIG. 5 is a perspective view of the objects of FIG. 1.

FIG. 6 is an expanded view of the distal portion of the objects of FIG. 5 at location C of FIG. 5.

FIG. 7 is a plan view of an arthroscopy ablator formed in accordance with the principles of this invention.

FIG. 8 is a side elevational view of the objects of FIG. 7.

FIG. 9 is a side elevational sectional view of the distal portion of the objects of FIG. 7 at location A-A.

FIG. 10 is a perspective view of the objects of FIG. 7.

FIG. 11 is an expanded view of the distal portion of the objects of FIG. 10 at location A of FIG. 10.

FIG. 12 is a plan view of an arthroscopy ablator formed in accordance with the principles of this invention, with the distal portion formed to a first angle.

FIG. 13 is a side elevational view of the objects of FIG. 12.

FIG. 14 is a side elevational sectional view of the distal portion of the objects of FIG. 12 at location A-A of FIG. 12.

FIG. 15 is a plan view of an arthroscopy ablator formed in accordance with the principles of this invention with the distal portion formed to a second angle.

FIG. 16 is a side elevational view of the objects of FIG. 15.

FIG. 17 is a side elevational sectional view of the distal portion of the objects of FIG. 15 at location A-A of FIG. 15.

FIG. 18 is a side elevational view of an arthroscopy ablator formed in accordance with the principles of this invention formed to a first angle, wherein the ablator is identical in all aspects to the previous embodiment except that the distal portion of the active electrode is angularly offset from the proximal portion of the active electrode.

FIG. 19 is an expanded elevational sectional view of the distal portion of the objects of FIG. 18 taken at the centerline.

FIG. 20 is a plan view of an alternate embodiment of the instant invention with the outer polymeric sleeve removed.

FIG. 21 is a side elevational view of the objects of FIG. 20.

FIG. 22 is an expanded view of the objects of FIG. 21 at location B.

FIG. 23 is a plan view of the objects of FIG. 20.

FIG. 24 is a sectional view of the objects of FIG. 23 at location A-A.

FIG. 25 is a plan view of the embodiment of FIG. 20 with the polymeric sleeve in place.

FIG. 26 is a sectional view of the objects of FIG. 25 at location A-A.

FIG. 27 is a plan view of the instant embodiment with the distal portion formed to a first angular offset.

FIG. 28 is a side elevational view of the objects of FIG. 27.

FIG. 29 is a sectional view of the objects of FIG. 27 at location A-A.

FIG. 30 is a plan view of a bending device for use with an electrosurgical device of the instant invention.

FIG. 31 is a perspective view of the objects of FIG. 30.

FIG. 32 is a side elevational view of the objects of FIG. 30.

FIG. 33 is an end view of the objects of FIG. 30.

FIG. 34 is a plan view of the bender of FIG. 30 in use applying an angular offset to the distal portion of an electrosurgical device of the present invention.

FIG. 35 is a perspective view of the objects of FIG. 34.

FIG. 36 is a sectional view of the distal portion of an alternate embodiment that is non-aspirating, wherein the embodiment is depicted with the distal portion angularly offset from the proximal portion.

FIG. 37 is a sectional view of an alternate non-aspirating embodiment in which the inner tubular member is replaced by a solid rod, wherein the embodiment is depicted with the distal portion angularly offset from the proximal portion.

FIG. 38 is an expanded side elevational view of the distal portion of an alternate embodiment electrosurgical device of the present invention.

FIG. 39 is a side elevational sectional view of the objects of FIG. 38.

FIG. 40 is an expanded side elevational view of the distal portion of another alternate embodiment device of the present invention.

FIG. 41 is a sectional view of the objects of FIG. 40 at location A-A.

FIG. 42 is a plan view of the distal portion of an alternate embodiment rebendable ablation device of the present invention.

FIG. 43 is a side elevational view of the objects of FIG. 62.

FIG. 44 is a sectional view of the objects of FIG. 42 at location A-A.

FIG. 45 is an expanded view of the sectional view of FIG. 44 at location C.

FIG. 46 is a plan view of the distal portion of an alternate embodiment rebendable ablation device of the present invention.

FIG. 47 is a sectional view of the objects of FIG. 46 at location A-A.

FIG. 48 is a plan view of the distal portion of an alternate embodiment rebendable ablation device of the present invention.

FIG. 49 is an expanded sectional view of the objects of FIG. 48 at location A-A.

### DETAILED DESCRIPTION OF THE PREFERRED EMBODIMENTS:

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims. Accordingly, unless otherwise defined, all technical and scientific terms used herein have the same meaning as commonly understood by one of ordinary skill in the art to which the present invention belongs. However, in case of conflict, the present specification, including definitions below, will control.

In the context of the present invention, the following definitions apply:

The words “a”, “an” and “the” as used herein mean “at least one” unless otherwise specifically indicated. Thus, for example, reference to an “opening” is a reference to one or more openings and equivalents thereof known to those skilled in the art, and so forth.

The term “proximal” as used herein refers to that end or portion which is situated closest to the user of the device, farthest away from the target surgical site. In the context of the present invention, the proximal end of the rebendable bendable endoscopic device includes the handle.

The term “distal” as used herein refers to that end or portion situated farthest away from the user of the device, closest to the target surgical site. In the context of the present invention, the distal end of the rebendable bendable endoscopic device includes one or more active electrodes end as well the regions of non-uniform flexural strength that permit bending.

In the context of the present invention, the term “cannula” is used to generically refer to the family of generally rigid, typically elongate lumened surgical instruments that facilitate access across tissue to an internally located surgery site.

The terms “tube” and “tubular” are interchangeably used herein to refer to a generally round, long, hollow component having at least one central opening often referred to as a “lumen”.

The terms “lengthwise” and “axial” are used interchangeably herein to refer to the direction relating to or parallel with the longitudinal axis of a device. The term “transverse” as used herein refers to the direction lying or extending across or perpendicular to the longitudinal axis of a device.

The term “lateral” pertains to the side and, as used herein, refers to motion, movement, or materials that are situated at, proceeding from, or directed to a side of a device.

The term “medial” pertains to the middle, and as used herein, refers to motion, movement or materials that are situated in the middle, in particular situated near the median plane or the midline of the device or subset component thereof.

The present invention contemplates repeatedly flexing, bending or angling the distal region of an endoscopic device to a wide range of angles relative to the longitudinal axis of the device, such angles ranging from greater than 0 to about 90 degrees, preferably from about 5 to about 60 degrees, more preferably from about 10 to about 45 degrees, even more preferably from about 10-20 to about 30-40 degrees.

To facilitate bending, the present invention contemplates providing the elongate distal tubular member of an electrosurgical device with a non-uniform flexural strength throughout its length. In the context of the present invention, the term “flexural strength”, also known as yield strength or bend strength, is a mechanical parameter of a material or component defined as its ability to resist inelastic transverse deformation under load that can be readily quantified and compared using conventional assays, such as the transverse bending test.

In the Examples below, the present invention makes reference to “notches” on opposing sides of a distal tubular member. In the context of the present invention, the term “notch” refers to a preferably long, narrow indentation, aperture, or incision disposed at an edge or surface. Although the notches described hereinbelow are depicted as generally “V-shaped” or “cylindrical” or “circular”, it will be readily apparent to the skilled artisan that the shape may be readily varied; for example, the notches may have a non-uniform and/or non-linear (i.e., curved) profile.

In the Examples below, respective sets of notches or apertures are preferably offset such that the notches on one side are centered between notches on the opposite side. It is these notches that serve to reduce the flexural strength of the device and thus permit bending in the distal region. However, as noted above, alternate mechanisms for reducing flexural strength are contemplated including, for example, reducing the wall thickness in the bend region, annealing the tube in the bend region, or by utilizing a material having lower flexular and/or yield strength to form the bend region.

The term “convex” is used herein to describe an element(s) that has a shape like the outside of a bowl, that is curved or rounded outward like the exterior of a sphere of circle. Alternatively, the term “concave” is used herein to describe an element that has a shape like the inside of a bowl, hollowed, curving or rounded inward like the inside of a sphere or circle. In the context of the present invention, when the device is flexed in a “downward” direction (relative to the longitudinal axis of the device), the “top” side that makes up the “outside” of the curve is referred to herein as the “convex side”, whereas the “bottom” side that makes up the “inside” of the bend is referred to as the “concave side”. The components are reversed when the device is flexed in an “upward” direction relative to the longitudinal axis of the device. In that case, the top side is the concave side and the bottom side is the convex side. As discussed in greater detail below, the present invention also contemplates flexing in the lateral plane.

The present invention makes reference to endoscopic electrosurgical devices, more particularly RF devices. However, the term “device” may be used interchangeably with the terms “instrument” and “probe”. Such electrosurgical devices typically include a “structural member”, “elongate portion” or “shaft” that directly conducts energy to the respective electrodes. The structural member is typically elongate, preferably conductive and more preferably formed of metal or metallic material. In certain embodiments, the elongate shaft may be hollow, including a lumen running therethrough that serves as a channel for a rigid inner element, an aspiration path for removing gaseous and liquid ablation byproducts, or an irrigation path for introducing preferably conductive irrigant to the target site. However, non-lumened and non-aspirating embodiments are also contemplated. The elongate shaft may optionally be electrically insulated by means of a dielectric coating, or, alternatively, via an external polymeric sleeve.

Electrosurgical devices contemplated by the present invention may be fabricated in a variety of sizes and shapes to optimize performance in a particular surgical procedure. For instance, instruments configured for use in small vascular spaces such as the brain may be highly miniaturized while those adapted for shoulder, knee and other large joint use may need to be larger to allow high rates of tissue removal. Likewise, electrosurgical instruments for use in arthroscopy, otolaryngology and similar fields may be produced with a rounded geometry, e.g., circular, cylindrical, elliptical and/or spherical, using turning and machining processes, while such geometries may not be suitable for other applications. Accordingly, the geometry (i.e., profile, perimeter, surface, area, etc.) may be square, rectangular, or polygonal or alternatively have an irregular shape suited to a specific need or anatomy.

The endoscopic electrosurgical instruments of the present invention are characterized by the presence of one or more elements referred to herein as “electrodes”. In certain embodiments, such electrodes are ring electrodes, preferably manufactured by machining from bar stock or hypodermic tubing, or, for other more complex geometries, more preferably formed by metal injection molding. Such molded electrodes may optionally be provided with features that locally increase the current density such as, for instance, arrays of grooves or protuberances. In the context of the present invention, the one or more electrodes are preferably fabricated from a suitable metallic material such as, for instance, stainless steel, nickel, titanium, molybdenum, tungsten, and the like as well as combinations thereof. However, electrically conductive non-metals are also contemplated.

In the context of the present invention, the “active electrode” is generally disposed at the distal end of the instrument. In the context of the present invention, the one or more electrodes are all connected, for example via wiring disposed within the control/handle portion of the instrument, to a power supply, for example, an externally located electrosurgical generator.

In certain embodiments, the present invention makes reference to one or more “insulators” separating the respective electrodes. As used herein, the term “insulator” refers to a electrically non-conductive element formed from a suitable dielectric material, examples of which include, but are not limited to, alumina, zirconia, and high-temperature polymers formed as solid, or non solid, such as fibers. Alternatively, the insulator may take the form of a dielectric coating utilized to cover portions of the electrode and leave others exposed. Suitable coatings may be from suitable polymeric materials applied, for instance, as a powder coat or liquid that is subsequently cured, or as a molded or extruded tube which is shrunk by heat after application.

Components of multi-electrode assembly may optionally be held in place by such coatings, although a suitable adhesive cement may also be used.

Like the overall electrosurgical instrument, the size, shape and orientation of the respective electrodes and the various active surfaces defined thereby may routinely vary in accordance with the need in the art. It will be understood that certain geometries may be better suited to certain utilities. Accordingly, those skilled in the art may routinely select one shape over another in order to optimize performance for specific surgical procedures. In addition, the electrodes may be formed and arranged in a variety of configurations to accomplish tissue vaporization for a range of applications and conditions. These include, but are not limited to, bulk tissue vaporization, tissue cutting, and producing holes in tissue.

In certain embodiments, the present invention makes reference to “conductive fluid(s)”, particularly in connection with the “wet environment” embodiments. As used herein, the term “fluid” encompasses liquids, gases and combinations thereof, either electrically conductive or non-conductive, intrinsic to the tissue or externally supplied. In the context of the present invention, the term “fluid” extends to externally supplied liquids such as saline as well as bodily fluids, examples of which include, but not limited to, blood, plasma, saliva, peritoneal fluid, lymph fluid, pleural fluid, gastric fluid, bile, and urine.

The present invention makes reference to the ablation, coagulation, vaporization and cauterization of tissue. As used herein, the term “tissue” refers to biological tissues, generally defined as a collection of interconnected cells that perform a similar function within an organism. Four basic types of tissue are found in the bodies of all animals, including the human body and lower multicellular organisms such as insects, including epithelium, connective tissue, muscle tissue, and nervous tissue. These tissues make up all the organs, structures and other body contents. The present invention is not limited in terms of the tissue to be treated but rather has broad application, including the resection and/or vaporization of any target tissue with particular applicability to the ablation, vaporization, destruction and removal of tissue in joints of the body as well as musculoskeletal applications.

The instant invention has both human medical and veterinary applications. Accordingly, the terms “subject” and “patient” are used interchangeably herein to refer to the person or animal being treated or examined. Exemplary animals include house pets, farm animals, and zoo animals. In a preferred embodiment, the subject is a mammal, more preferably a human.

Hereinafter, the present invention is described in more detail by reference to the Figures and Examples. However, the following materials, methods, figures, and examples only illustrate aspects of the invention and are in no way intended to limit the scope of the present invention. For example, while the present invention makes specific reference to arthroscopic procedures, it is readily apparent that the teachings of the present invention may be applied to other minimally invasive procedures and thus are not limited to arthroscopic uses. As such, methods and materials similar or equivalent to those described herein can be used in the practice or testing of the present invention.

### EXAMPLES

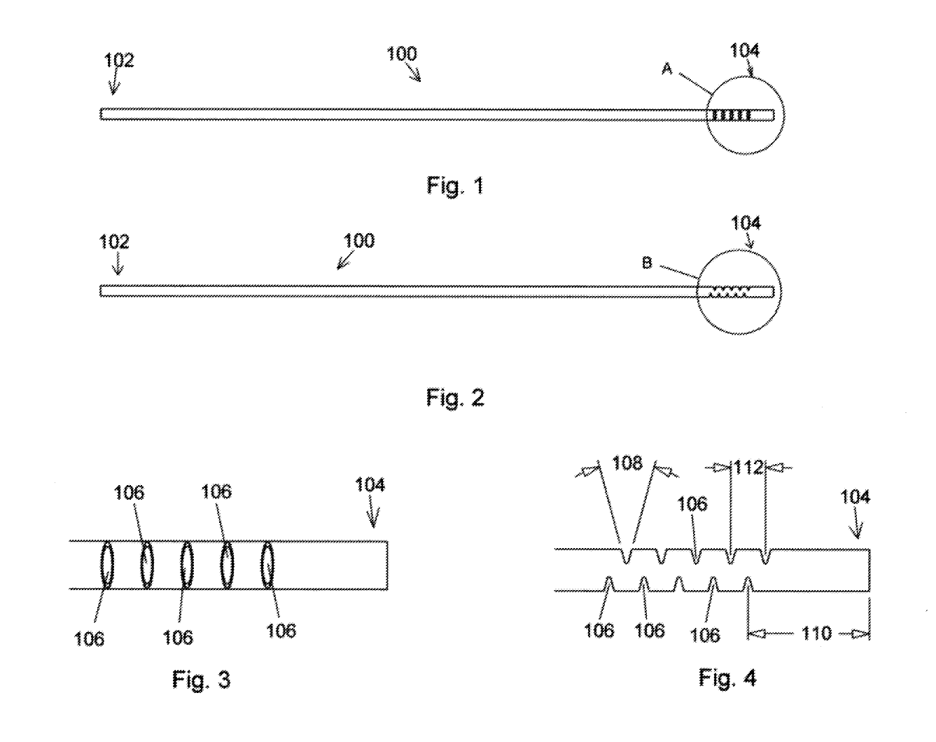

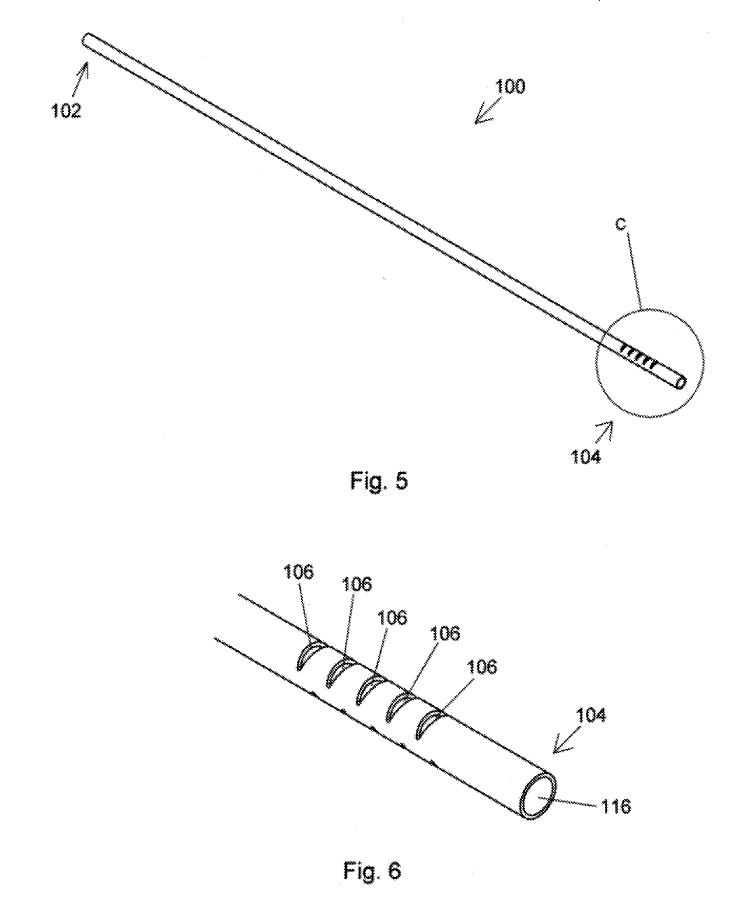

FIGS. 1 through 6 depict a tubular member 100 forming the distal end of a rebendable arthroscopy ablator in accordance with the principles of this invention. Tubular member 100 has a proximal end 102, a distal end 104 and inner lumen 116 extending therebetween. The portion of the tubular member 100 proximal to distal end 104 is provided with angular slots 106 of included angle 108 formed therein, wherein slots 106 at their distal limit are displaced distance 110 from the distal end 104. Slots 106 are spaced from each other by distance 112. Slots on opposing sides of the tube are offset such that the slots 106 on one side are centered between slots 106 on the opposite side. In use, the respective opposing sides are referred to as the “convex side”, which makes up the “outside” of the bend, and the “concave side”, which makes up the “inside” of the bend.

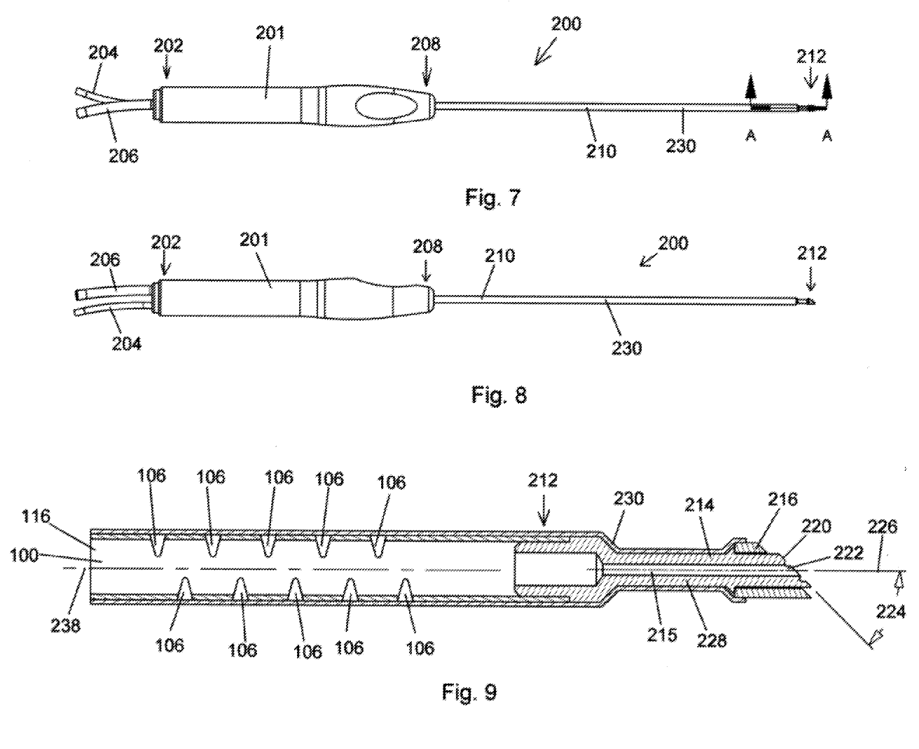

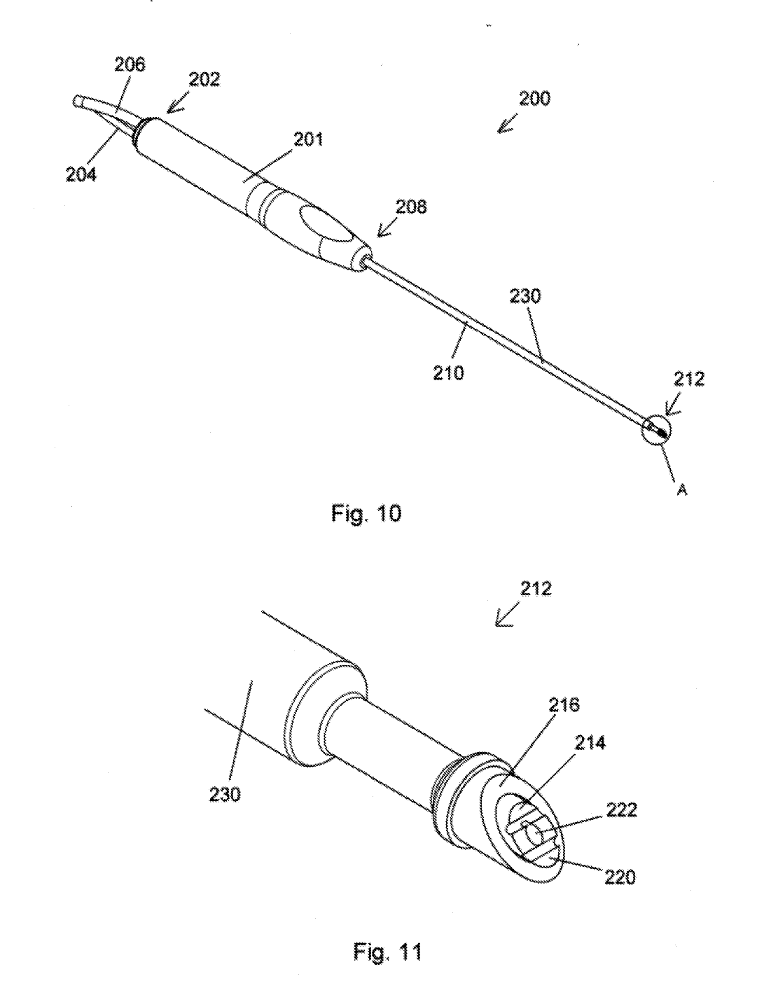

Referring now to FIGS. 7 through 11, which depict a rebendable aspirating arthroscopy ablator 200 formed in accordance with the principles of this invention, ablator 200 has a proximal portion forming a handle 201 having a proximal end 202 from which pass cable 204, which is configured for connection to an electrosurgical generator (not shown), and tubular member 206, which is configured for connection to an external vacuum source (not shown). Handle 201 has a distal end 208 connected to tubular distal portion 210 which has at its distal end 212 a distal assembly consisting of electrode element 214 and insulator 216. Tubular distal portion 210 formed with tubular member 100 is covered with a dielectric coating 230 which extends from distal end 208 of handle 201 to the proximal end of insulator 216 such that only the distal-most surface 220 of electrode element 214 and the immediately surrounding protruding portion of electrode element 214 are uninsulated. Distal-most surface 220 of electrode element 214, with its associated optional grooves, forms an ablating surface, RF energy being conducted to electrode 214 by cable 204, means with handle 201, and tubular element 100. Aspiration port 222 formed in surface 220, in communication with lumen 116 of tubular member 100 through lumen 215 of electrode element 214, and means within handle 201 and tubular element 206 provides a flow path for aspirating ablation byproducts from the site during use. Distal-most surface 220 forms an angle 224 with the longitudinal axis 226 of the part of distal tubular distal portion 210 that is adjacent to distal end 212. By bending mid-portion 218 of electrode element 214 and varying angle 224, distal-most surface 220 may be oriented at any angle from zero to 90 degrees with respect to the axis 238 of distal portion 210.

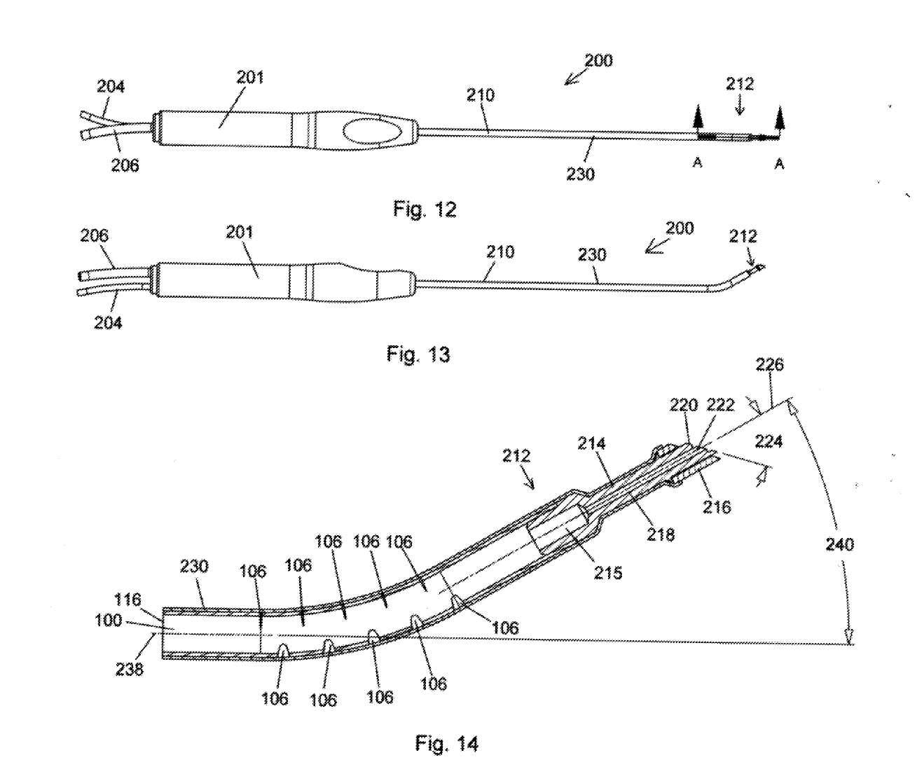

FIGS. 12 through 14 depict a rebendable ablator 200 formed in accordance with the principles of the present invention, with its distal portion bent to a first angle 240 with respect to the longitudinal axis 238 of distal portion 210. Distal-most ablating surface 220 is oriented at a first angle with respect to longitudinal axis 238 of distal portion 210, the first angle being determined by angle 224 and angle 240. The distal portion may be continuously bent until notches 106 on the concave side of the bend are closed up, with angle 240 representing the limit to which the distal portion of tubular member 100 may be bent. Notches 106 on the convex side are spread to larger angles due to the bending action.

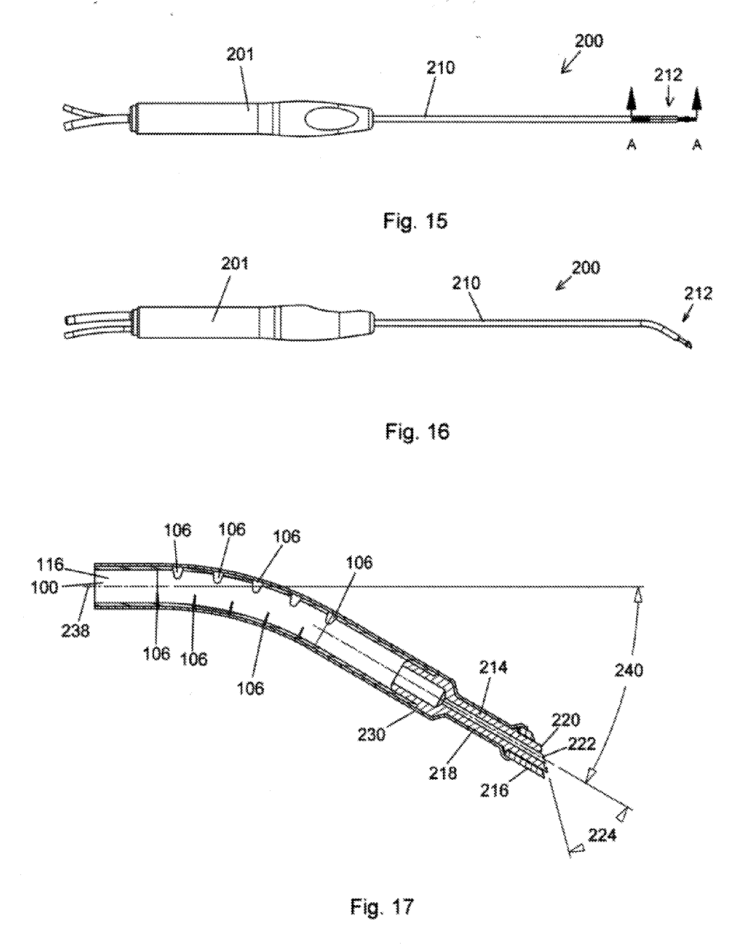

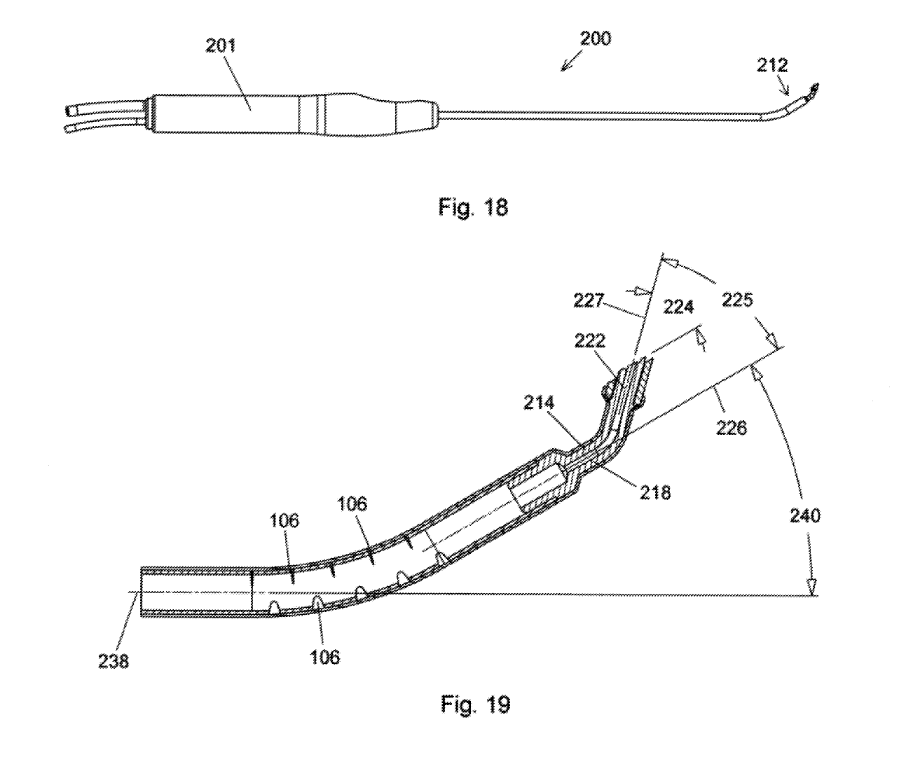

FIGS. 15 through 17 again depict rebendable ablator 200, this time with its distal portion rebent to a second angle 240 with respect to longitudinal axis 238 of more proximal portions of distal portion 210. Distal-most ablating surface 220 is oriented at a second angle with respect to axis 238 of distal portion 210, the second angle being determined by angle 224 and angle 240. Again, the distal portion may be continuously bent until notches 106 on the concave side of the bend closed up, with angle 240 again representing the limit to which the distal portion of tubular member 100 may be bent. The included angle of notches 106 on the convex side is increased due to the bending action.

By making available to surgeons rebendable devices 200 in which angle 224 may have a range of values, and by allowing the surgeons to over and over bend and rebend these devices 200 to a range of angles, surgeons are able to treat tissues at difficult to reach locations in a single procedure, using a single device, simply by adjusting the angular offset of the distal portion of devices 200. As such, a rebendable device in accordance with the present invention affords maximal access to various tissues during a procedure.

Notches 106 in tubular member 100 are laterally opposed so that the distal portion of tubular member 100 may be bent either upward or downward, relative to the longitudinal axis 238 of the distal portion 210. In other embodiments, notches 106 may be positioned on only one side of the tubular member, with notches on the top surface allowing upward bending only, and notches on the bottom side allowing downward bending only. Alternatively, in some cases, it may be desirable to have the bends in the lateral plane of the device, a configuration achieved by positioning the notches in that plane. The present invention contemplates a multitude of notch positions and deems all to fall within the scope of this invention since all are produced by locally reducing the flexural strength of tubular member 100.

While the rebendable RF device 200 of the embodiment previously herein described has a tip configured for bulk vaporization of tissue, other electrode configurations may be used without departing from the principles of this invention. These configurations include, for example, various hooks, needles and blade-like electrodes, or electrodes that combine, for instance, the ability to cut with the ability to efficiently accomplish bulk vaporization of tissue. Additionally, the electrodes may be configured for the thermal treatment of tissue.

The rebendable RF device 200 of the previously described embodiment is configured for use in a fluid filled cavity as is typically required of arthroscopy. Other embodiments are contemplated for use in dry or semi-dry environments, wherein the devices optionally incorporate an irrigation means as well as an aspiration means, or solely an irrigation means. Aspiration and irrigation may occur through separate lumens or, alternatively, may be alternatively performed using a single lumen.

The rebendable RF device 200 of the previously described embodiment uses a tubular member having non-uniform flexural strength provided by notching that portion of tubular member 100 that is to be bent. In other embodiments, the localized reduction in flexural strength may be achieved by reducing the wall thickness of the tube in the region, or by locally annealing the region, as with, for instance, an induction heater. Alternatively, the localized reduction in flexural strength may be achieved by utilizing a material having a lower flexular and/or yield strength to form the bend region.

Devices of the present invention previously herein described may be used with a remotely located return electrode, a construction referred to in the art as “monopolar”. In the context of a monopolar electrosurgical device, energy flows from the electrosurgical generator through cabling, handle, tubular member and ultimately an active electrode located at the distal region of the device to the patient, at which point it travels through the patient's body, and returns to the generator via a return electrode remotely located on the patient (for example, via a return pad positioned on the patient's skin). The principles of the instant invention may also be applied to electrosurgical devices that include a return electrode on the device itself, in proximity to the active electrode, a construction referred to in the art as “bipolar”. The construction of bipolar devices is similar to that of monopolar embodiments previously described except that a metallic elongate tubular member is coaxially positioned about tubular member 110 and separated therefrom by dielectric coating 210. The exterior surface of this member is covered by a dielectric polymeric sleeve except for an uninsulated distal region which functions as the return electrode, with the proximal end of the outer tubular member being connected by means within the device handle 201 and cable 204 to the electrosurgical generator so as to provide a return path for RF energy. The flexural strength of the distal portion of these bipolar devices is determined by the flexural strength of the assembly of the inner tubular member 100 and an outer tubular member that forms the return path. In bipolar embodiments of the present invention, distal slots 106 in tubular member 100 (FIGS. 1 through 4) are eliminated. The flexural strength of the outer tubular member and thereby the distal assembly is locally reduced by slots formed in its distal portion of the outer tube. The flexural strength and rigidity are determined by flexural strength of the assembly of the un-slotted inner tubular member 100 and the outer tubular member with its distal slots.

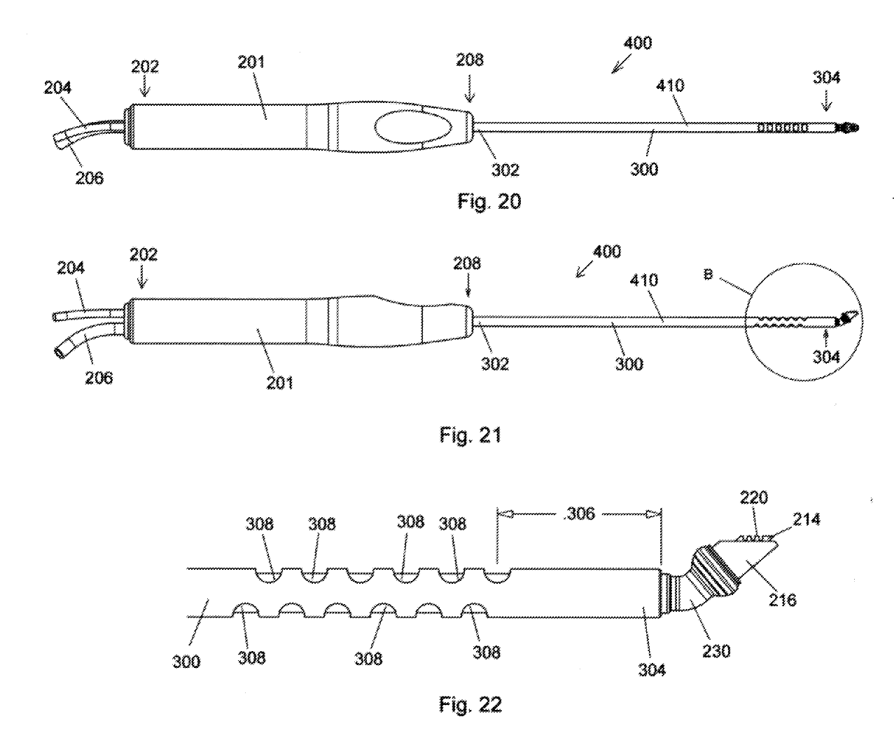

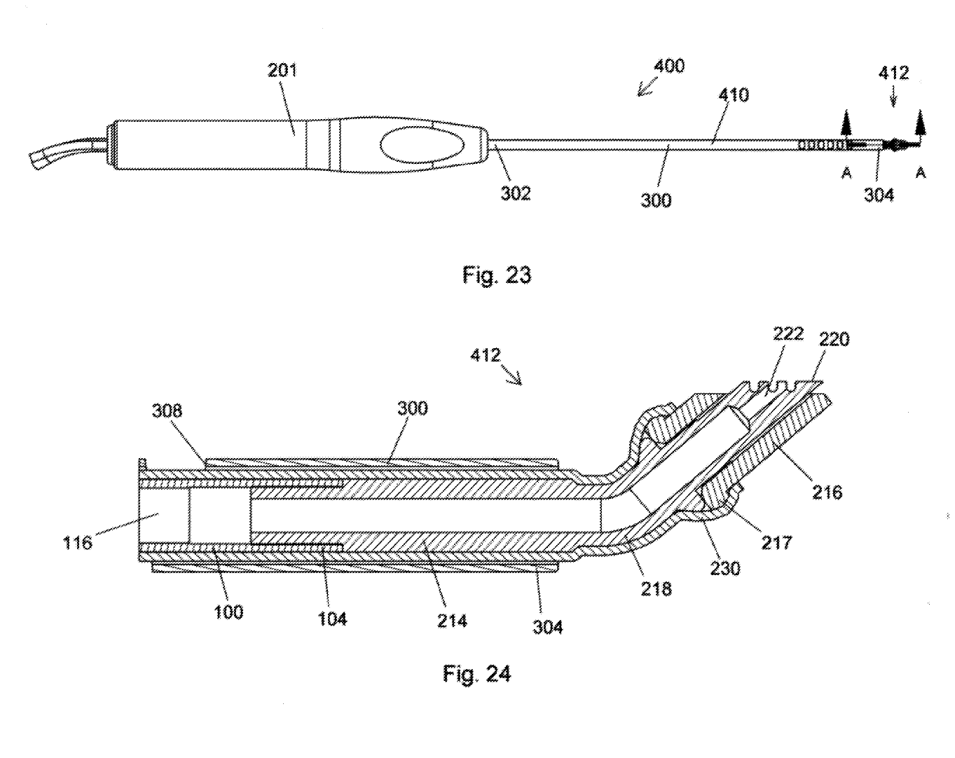

FIGS. 20 through 24 depict a rebendable endoscopic ablation device 400 with elongate distal portion 410 having an uninsulated region 312 that functions as a return electrode on the device in proximity to the active electrode 214, but with an external polymeric sheath removed to better reveal details of the construction. Device 400 differs from device 200 only in the construction aspects subsequently described. Distal slots 106 in tubular member 100 are eliminated, and tubular member 300 is coaxially positioned about tubular member 100 and separated and electrically isolated from tubular member 100 by dielectric coating 230. In assembly, the outer tubular member 300 is preferably slidably disposed over the inner tubular member 100 or vice versa. Their respective proximal ends are then both rigidly affixed at the handle so as to preclude relative axial movement of the two members during use.

Tubular member 300 has formed distance 306 from its distal end 304 notches 308. Slots on opposing sides of the tubular member 300 are offset such that the notches 308 on one side are centered between notches 308 on the opposite side. Proximal end 302 is connected by means within handle 201 to cable 204 and therethrough to the return receptacle of the electrosurgical generator (not shown). Notches 308 of tubular member 300 function in the same manner as slots 106 of tubular member 100 of device 200; that is, they are formed in tubular member 300 to locally reduce the flexural strength of tubular member 300.

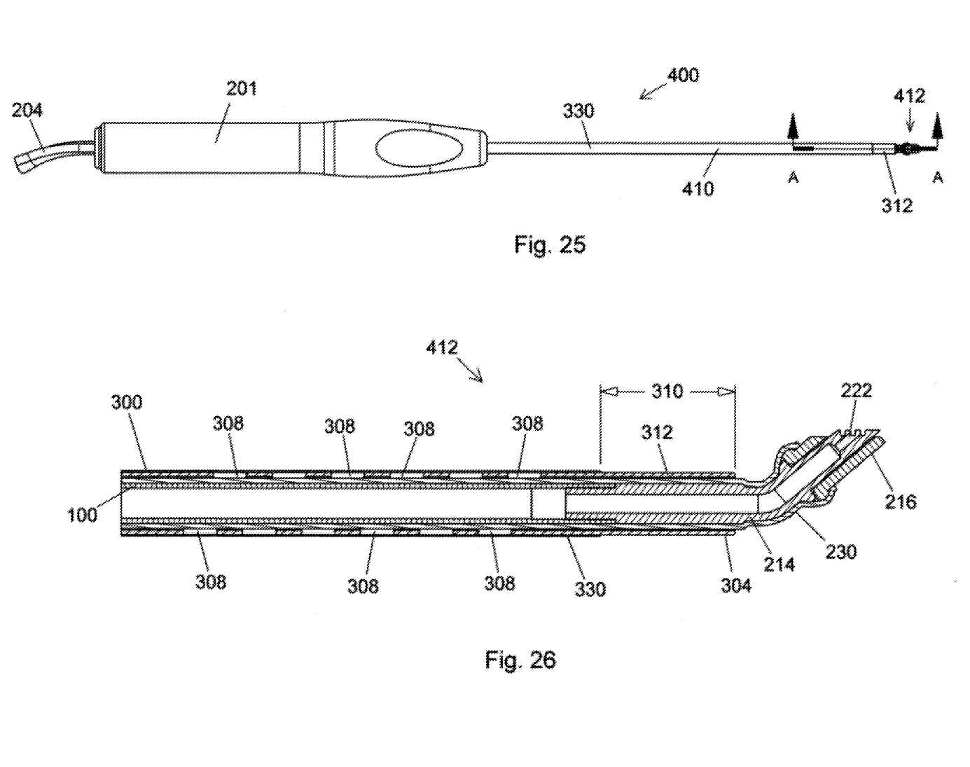

FIGS. 25 and 26 depict device 400 with polymeric sleeve 330 covering the external surface of tubular member 300 from handle 201 at the proximal end of tubular member 300 to distance 310 from distal end 304 so as to leave uninsulated region 312. During use, uninsulated region 312 functions as a return electrode providing a path via tubular member 300, means within handle 201, and cable 204 for energy to return to the electrosurgical generator. When using device 400, a remote return electrode is not connected to the generator.

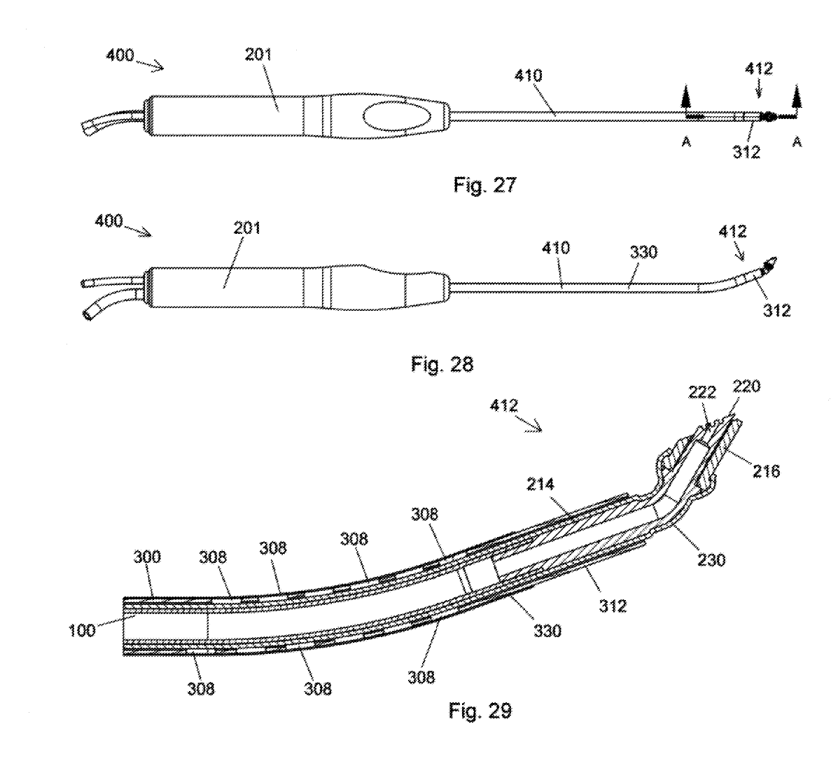

In FIGS. 27 through 29, the distal portion of tubular distal portion 410 is angularly offset, tubular member 300 and tubular member 100 being bent in the region of notches 308 in tubular member 300 where the flexural strength of tubular member 300 is locally reduced.

The rigidity of endoscopic devices must be sufficient to prevent deformation during use. This requirement is especially important when the devices have elongate distal portions of extended length, like those used for endoscopic hip surgery. Accessing a site within a hip during surgery frequently requires that the surgeon apply significant force to the handle of the device to overcome resistance by tissue surrounding the distal portion of the device. If the distal portion of the device has insufficient flexural strength, it will deform and access to certain tissue structures may be prevented or limited. Accordingly, the design of extended length endoscopic devices must provide distal portions with high flexural strength. Device 400 has a high flexural strength provided by the construction of distal portion 410 with its coaxial rigid metallic tubular elements separated by dielectric coating 230. While the flexural strength of distal portion 410 is locally reduced through the inclusion of slots 308 in tubular member 300, significant flexural strength remains since tubular member 100 is not notched and retains its flexural strength. Notches 308 in tubular member 300 localize bending and rebending in the region of notches 308. However, such bending may require that a significant bending moment be applied. In such cases, an external bending tool may be required.

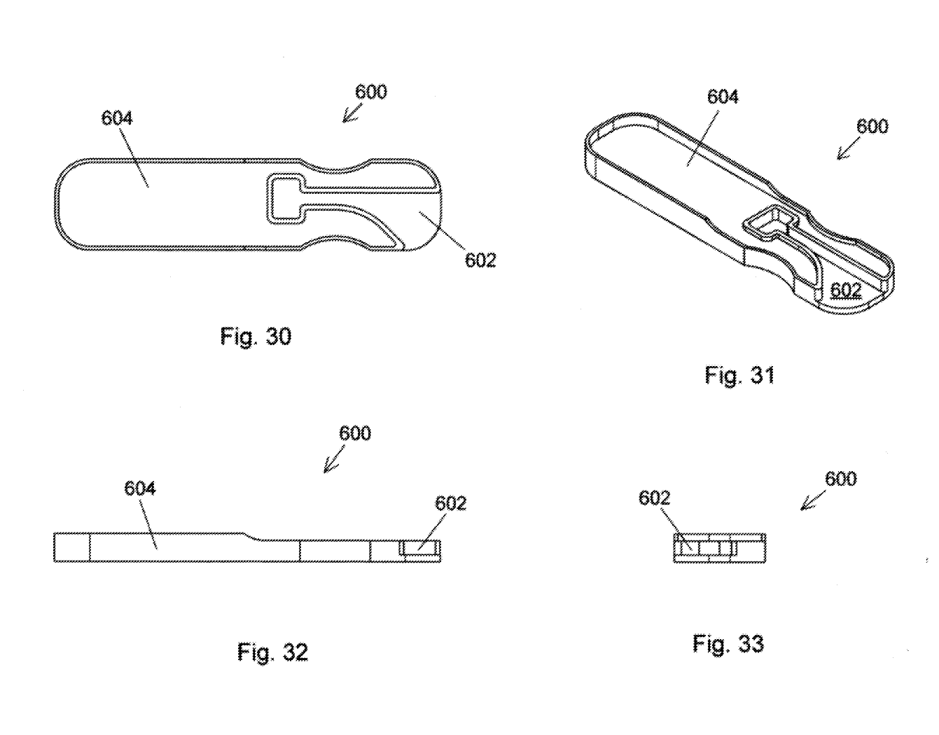

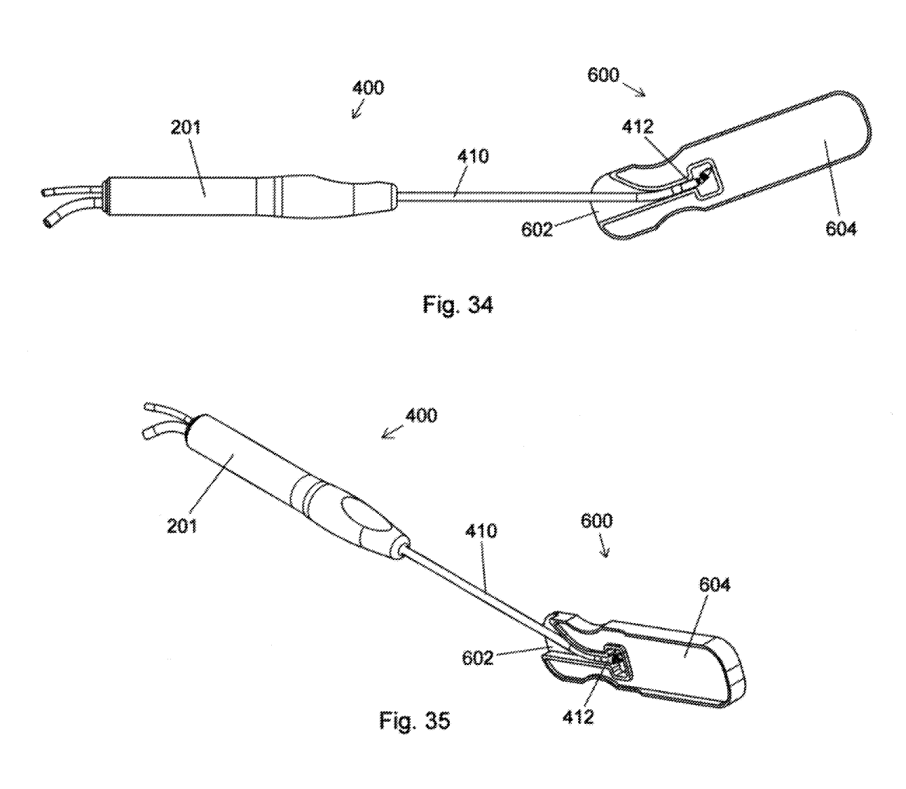

An illustrative bending element 600 for applying the required moment to device 400 or device 200 is depicted in FIGS. 30 through 33. Bender 600 has a distal pocket 602 configured to accept the distal portion of elongate distal portion 410 of device 400, and a proximal handle portion 604. As depicted in FIGS. 34 and 35, wherein device 400 and bender 600 are positioned for bending of distal portion 410 of device 400, leverage provided by the configuration of bender 600 with its substantial handle portion allows the distal portion 410 of device 400 to be readily bent to a first angular offset, straightened and/or bent to a second angular offset, and rebent as required, with distal portion 410 of device 400 having the required rigidity for effective use by the surgeon. Indeed, the rigidity of distal portion 410 is primarily determined by the rigidity of the coaxial assembly of tubular member 100 with dielectric coating 230 and the notched portion of tubular member 300. Notches 308 in tubular member 300 locally reduce the flexural strength of tubular member 300 so as to localize bending and rebending in that region and to reduce the amount of “spring back” when bending distal portion 410. It is not necessary to reduce the flexural strength below that required for optimal surgeon use since bender 600 allows the surgeon to apply a significant bending moment.

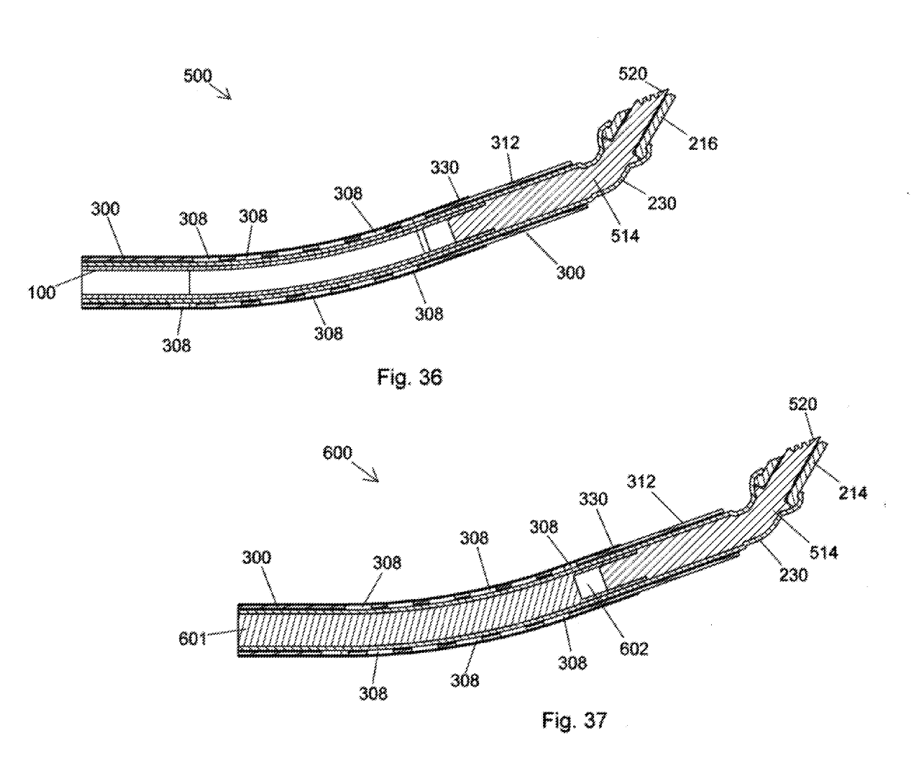

Embodiments previously herein described have an aspiration path from the ablating surface to an external vacuum source. In other embodiments, this aspiration path may be eliminated since for many procedures aspiration of bubbles and ablation byproducts is not required. Sectional views of the distal portions of two such embodiments are depicted in FIGS. 36 and 37. Device 500 (FIG. 36) is identical in all aspects to device 400 except that electrode 514 does not have a lumen, and tubular member 206 and its associated flow path through handle 201 (see FIGS. 20 and 21) are eliminated. Device 600 (FIG. 37) is identical to device 500 in all aspects except that tubular member 100 has been replaced by metallic rod 601 in which is formed distal recess 602 for the mounting thereto of electrode 514. The diameter and material properties of rod 601 may be optimized to provide required rigidity of the distal portion of device 600.

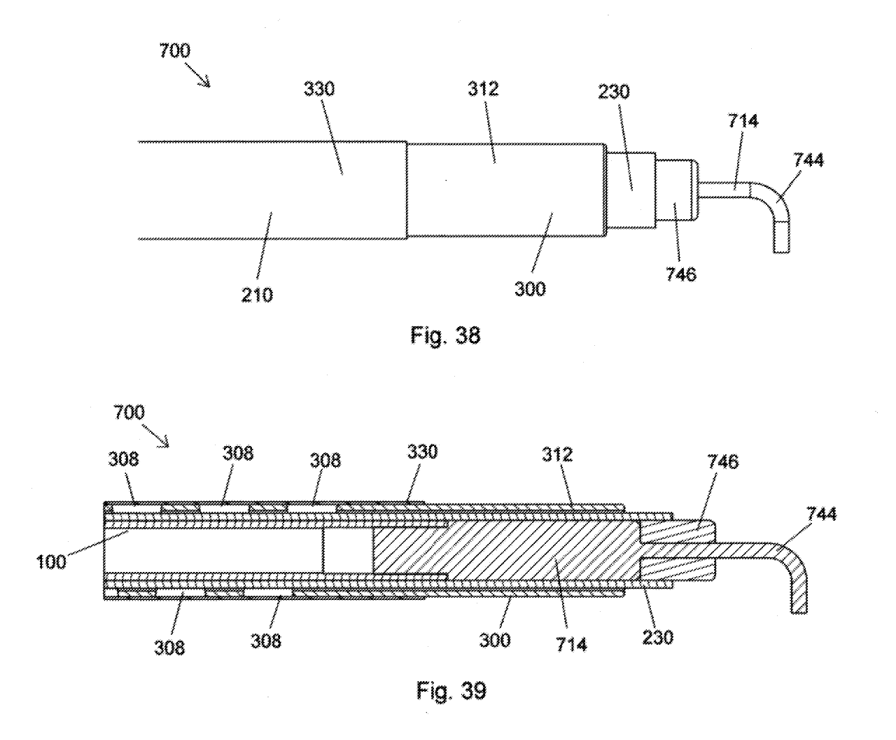

The principles of the instant invention may be advantageously applied to other electrosurgical devices that are not configured for bulk tissue vaporization as in the previous embodiments. For instance, FIGS. 38 and 39 depict the distal portion of an electrosurgical device 700 for the cutting of tissue using an active electrode 714 having a hook-shaped distal portion 744 that protrudes beyond insulator 746. Aspiration is not required, so in all aspects except for the configuration of electrode 714 and insulator 746, device 700 is identical to device 500. With regard to bending, straightening and rebending, device 700 functions in the same manner as previous embodiments.

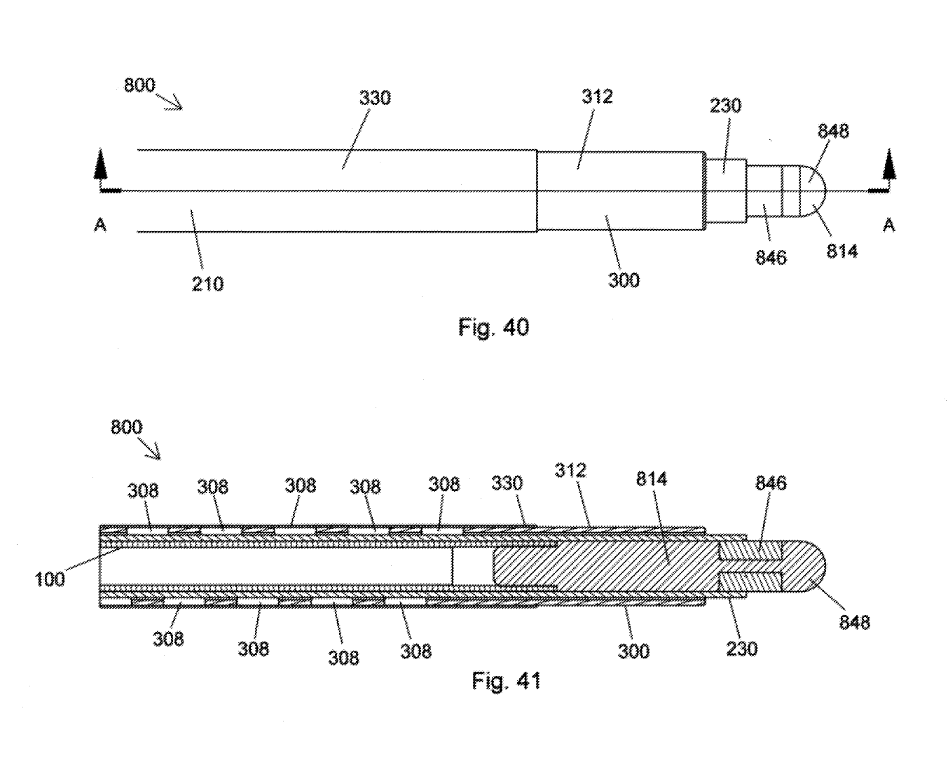

FIGS. 40 and 41 depict the distal portion of an electrosurgical device 800 of the present invention that is configured for the thermal treatment of tissue. Active electrode 814 has a rounded distal portion 848 distal to insulator 846 formed in a manner that minimizes regions of energy intensification so as to allow the desiccation of tissue without vaporization. Except for electrode 814, in all other aspects the construction of device 800 is identical to that of device 700. With regard to bending, straightening and rebending, device 800 functions in the same manner as previous embodiments.

In the above-described embodiments, RF energy is conducted to the distal end electrode piece by means of a structural element of the device to which it is affixed, i.e., either metallic tubular element 100 (see, e.g., devices 200, 400, 600 and 700) or metallic rod 601 (see, e.g., device 600), wherein conductive structural element is also contributing to the flexular strength (rigidity) of the device. For example, the rigidity of monopolar device 200 is determined solely by the rigidity of member 100. Similarly, in the case of bipolar devices 400, 600 and 700, the rigidity is determined by the rigidity of the elongate distal assembly including elongate metallic elements 100 or 601, and tubular member 300 and by the combined rigidity of these elements.

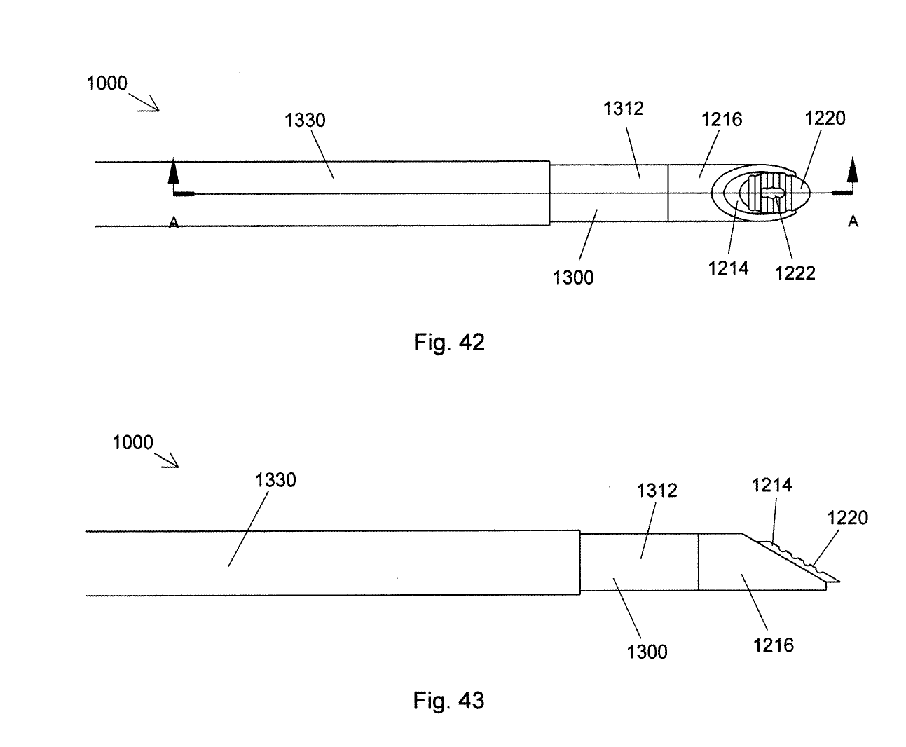

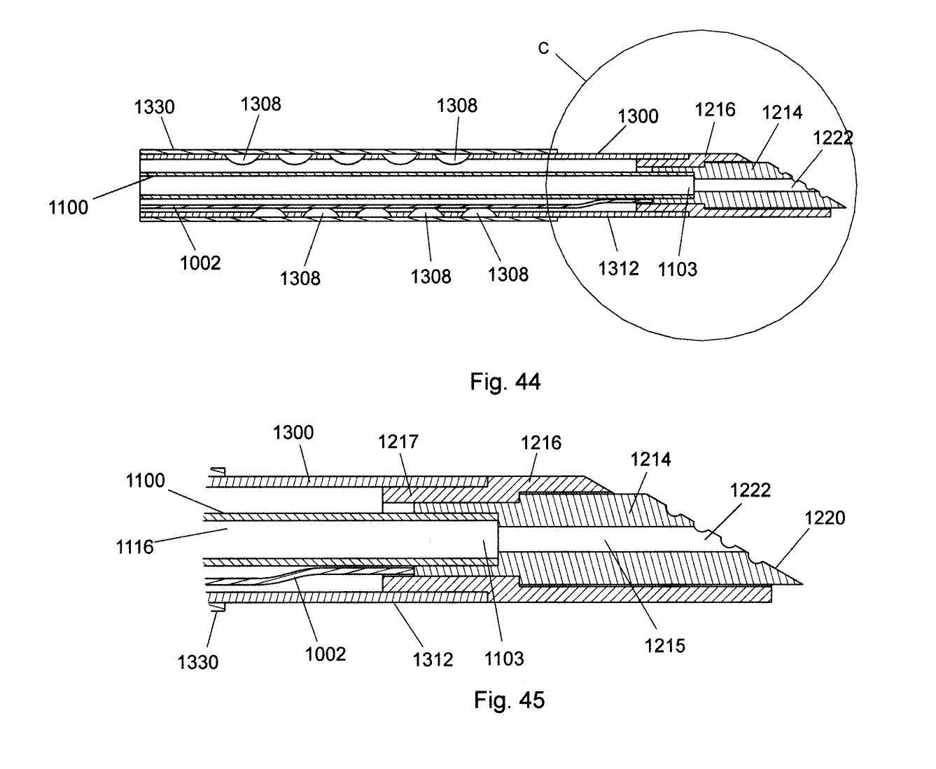

However, the present invention contemplates embodiments wherein RF energy is conducted to the electrode piece not by a structural element with flexular strength, but rather by a small diameter elongate conductive element such as, for instance, a wire having negligible rigidity. Referring now to FIGS. 42 through 45, alternate embodiment aspirating electrosurgical device 1000 is alike in all aspects of form and function to device 400 (FIGS. 20 through 29) except as specifically described hereinafter. Insulator 1216 is affixed to the distal end of outer tube 1300, proximal portion 1217 of insulator 1216 being configured for fixation thereto. Electrode element 1214 is positioned within insulator 1216 and is affixed thereto so that electrode piece 1214 is electrically isolated from tubular member 1300. Electrode piece 1214 is electrically connected by elongate conductive element 1002 and means within handle 201 to cable 204 (FIGS. 7 and 8) and thereby to a suitable source of RF energy. Tube 1100 is formed of a flexible polymeric material, distal end 1103 of tube 1100 being affixed to electrode piece 1214 as depicted in FIG. 45. Aspiration port 1222 formed in surface 1220, in communication with lumen 1116 of tubular member 1100 through lumen 1215 of electrode element 1214, and means within handle 201 and tubular element 1206 (FIGS. 7 and 8) provide a flow path for aspirating ablation byproducts from the site during use. The rigidity of device 1000 is determined solely by the rigidity of tubular element 1300.

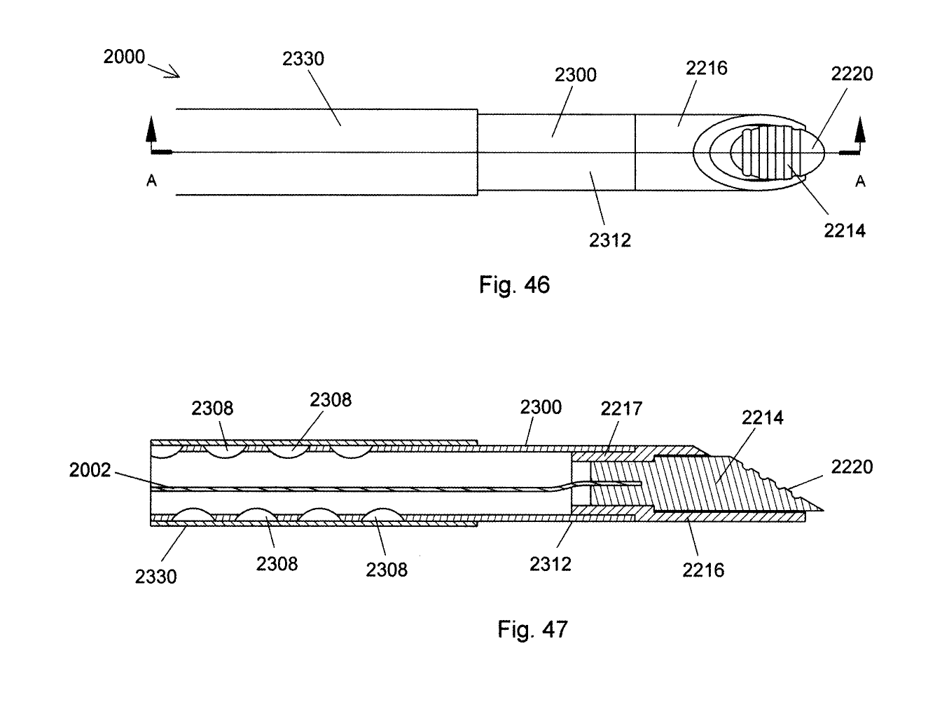

A non-aspirating embodiment is depicted in FIGS. 46 and 47, wherein device 2000 is identical to device 1000 in form and function except as specifically described hereinafter. Elements of the aspiration path of device 1000 are eliminated. RF energy is supplied to electrode element 2214 by elongate conductive element 2002.

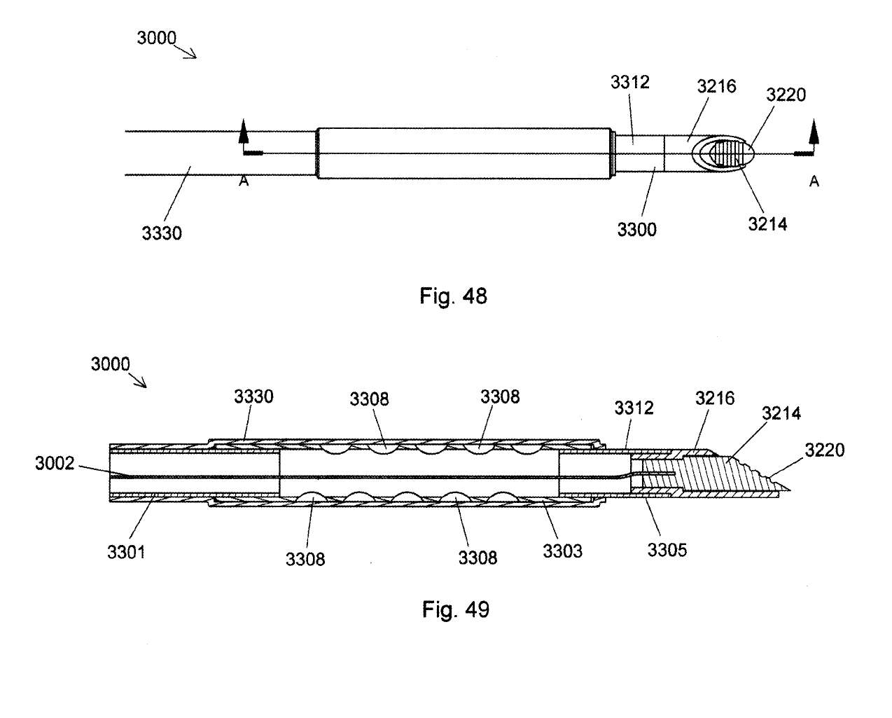

Another embodiment of the present invention is depicted in FIGS. 48 and 49. Device 3000 is identical to device 2000 in all aspects of form and function except as specifically described hereafter. For example, elongate tubular member 2300 of device 2000 may be replaced by a coaxial assembly of tubular elements, including proximal element 3301, distal element 3305, and middle element 3303 that is affixed to and links elements 3301 and 3305. Notches 3308 may be formed in middle element 3303. In certain embodiments, middle element 3303 may be formed from the same metallic material as proximal element 3301 and distal element 3305. However, in other embodiments, middle element 3303 may be formed from an alternate and/or dissimilar metallic material. For example, middle element 3303 may formed from a metallic material having a lower flexular or yield strength than the material from which proximal element 3301 and distal element 3305 are formed. In such embodiments, wherein a material having lower flexular and/or yield strength is utilized, slots 3308 may be optionally eliminated.

Devices 1000, 2000, and 3000 are operated in the same manner as the previous embodiments. Namely, the surgeon may apply a first suitable angular offset to the distal portion to vaporize or thermally treat tissue at a first location, and then apply a second angular offset of the distal portion to vaporize or thermally treat tissue at a second location, the process being repeated as required to complete a surgical procedure.

### INDUSTRIAL APPLICABILITY

As noted previously, there is a need in the endoscopic arts for electrosurgical devices that may be repeatedly flexed in the field to allow access to a wide array of remote tissues during a single minimally invasive procedure and using a single device. Devices of the present invention address this need and allow the surgeon to angularly rigidly offset the distal portion of the device for optimal access to tissue structures when performing endoscopic surgery. The distal portions of devices of the present invention may be bent and rebent with the bends located in a predetermined distal region of the device in which the flexural strength has been reduced. The rigidity of the devices is sufficient to allow use without deformation that would prevent optimal access. Bending of the devices is limited to a single plane, preferably a vertical plane along the centerline of the device, although embodiments that allow lateral bending are also anticipated. Although described in detail with respect to arthroscopic applications, it will be readily apparent to the skilled artisan that the utility of the present invention extends to other minimally invasive endoscopic devices and procedures.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

The invention has been illustrated by reference to specific examples and preferred embodiments. However, it should be understood that the invention is intended not to be limited by the foregoing description, but to be defined by the appended claims and their equivalents

What is claimed:

1. A bendable and rebendable electrosurgical device comprising a proximal handle portion assembled to an elongate distal portion that defines the longitudinal axis of the device, wherein said elongate distal portion comprises:
a. an elongate tubular member fabricated from a metallic material and having a proximal end configured for attachment to the distal end of said handle portion and a distal end that includes a bend region disposed in close proximity to said distal end and that comprises a length of reduced flexural strength relative to that of the remainder of the elongate tubular member, wherein said bend region may be repeatedly flexed away from said longitudinal axis, in the longitudinal or lateral direction;
b. a distal end assembly including an active electrode; and
c. a means for conducting RF energy to said active electrode.

2. The bendable and rebendable electrosurgical device of claim 1, wherein said means for conducting RF energy to said active electrode is said elongate tubular member.

3. The bendable and rebendable electrosurgical device of claim 1, wherein said means for conducting RF energy to said active electrode is a rigidly bendable element positioned within the lumen of said elongate tubular member.

4. The bendable and rebendable electrosurgical device of claim 1, wherein said means for conducting RF energy to said active electrode is a tubular element.

5. The bendable and rebendable electrosurgical device of claim 4, wherein said tubular element further provides a flow path for aspirating ablation byproducts from the surgical site during use.

6. The bendable and rebendable electrosurgical device of claim 1, wherein said means for conducting RF energy to said active electrode is a wire.

7. The bendable and rebendable electrosurgical device of claim 1, wherein material in the bend region has a reduced yield strength relative to that of the remainder of the elongate tubular distal member that serves to reduce the overall flexural strength of said bend region relative to that of the remainder of the elongate tubular member.

8. The bendable and rebendable electrosurgical device of claim 1, wherein bend region is fabricated from a first material that has a reduced flexular strength relative to a second material from which the remainder of elongate tubular member is fabricated.

9. The bendable and rebendable electrosurgical device of claim 8, wherein said elongate tubular member is a coaxial assembly of discrete proximal, middle, and distal elements, wherein a distal end of said proximal element is affixed to a proximal end of said middle element, a distal end of said middle element is affixed to a proximal end of said distal element, and a distal end of said distal element is affixed to said distal end assembly that includes the active electrode, further wherein said proximal and distal elements are fabricated from said second material and said middle element is fabricated from said reduced flexular strength first material.

10. The bendable and rebendable electrosurgical device of claim 1, wherein bend region has a reduced wall thickness relative to that of the remainder of the elongate tubular distal member that serves to reduce the overall flexural strength of said bend region relative to that of the remainder of the elongate tubular member.

11. The bendable and rebendable electrosurgical device of claim 1, wherein said elongate tubular member further comprises an insulating polymeric sleeve or dielectric coating extending over said elongate tubular member, from its proximal end to just past said bend region, thereby defining an uninsulated distalmost region disposed between the distal end of said bend region and said active electrode that acts as a return electrode.

12. The bendable and rebendable electrosurgical device of claim 1, wherein said repeated flexing comprises (a) bending said distal portion to a first angle, and (b) rebending said distal portion to a second angle, wherein said first angle enables contact of the active electrode to a first set of target tissues and said second angle enables contact of the active electrode to a second set of target tissues.

13. The bendable and rebendable electrosurgical device of claim 1, wherein said first angle extends in a first direction and said second angle extends in a second opposed direction.

14. The bendable and rebendable electrosurgical device of claim 1, wherein said first and second angles extend in the same direction but are not equivalent.

15. The bendable and rebendable electrosurgical device of claim 1, wherein bend region comprises a plurality of notches extending from the outer surface of said tubular member to the lumen of said tubular member that serve to reduce the overall flexural strength of said bend region relative to that of the remainder of the elongate tubular member.

16. The bendable and rebendable electrosurgical device of claim 15, wherein said plurality of notches comprises (a) a first series of notches disposed on a top side of said bend region and (b) a second series of notches disposed on an opposed bottom side of said bend region.

17. The bendable and rebendable electrosurgical device of claim 16, wherein said second series of notches is offset from and centered between said first series of notches.

18. The bendable and rebendable electrosurgical device of claim 15, wherein said plurality of notches comprises a generally V-shaped incision disposed in the surface of said bend region.

19. The bendable and rebendable electrosurgical device of claim 15 wherein each of said plurality of notches comprises a generally arcuate incision disposed in the surface of said bend region.

20. The bendable and rebendable electrosurgical device of claim 1, wherein said elongate conductive element and said elongate tubular member are separated by an insulating polymeric sleeve or dielectric coating.
